(19) United States
(12) Patent Application Publication
(10) Pub. No.: US 2026/0103505 A1
(43) Pub. Date: Apr. 16, 2026

(54) INTEGRIN BETA 2 (CD18) GENE-EDITED BOVINE CELLS AND ANIMALS WITH REDUCED SUSCEPTIBILITY TO MANNHEIMIA HAEMOLYTICA LEUKOTOXIN A (LKTA)

(71) Applicant: The United States of America, as represented by the Secretary of Agriculture, Washington, DC (US)

(72) Inventors: MICHAEL P. HEATON, Clay Center, NE (US); Aspen M. Workman, Clay Center, NE (US)

(21) Appl. No.: 19/302,270

(22) Filed: Aug. 18, 2025

Related U.S. Application Data
(60) Provisional application No. 63/686,289, filed on Aug. 23, 2024.

(51) Int. Cl. C07K 14/705; A01K 67/0275

(52) U.S. Cl. CPC C07K 14/70553; A01K 67/0275; A01K 2217/072; A01K 2227/101

(57) ABSTRACT

The instant disclosure provides multiple precision edits of bovine integrin beta 2 (CD18) that confer reduced susceptibility to Manheimia haemolytica leukotoxin A (LktA). Further disclosed are methods of producing genome-edited bovine cells, embryos, and animals comprising a modified CD18 incorporating the precision edits.

### CROSS-REFERENCE

The present application claims priority to U.S. Provisional Patent Application Ser. No. 63/686,289 filed Aug. 23, 2024, the content of which is expressly incorporated herein by reference.

### SEQUENCE LISTING

The instant application contains a Sequence Listing XML required by 37 C.F.R. § 1.831(a) which has been submitted in XML file format via the USPTO patent electronic filing system and is hereby incorporated by reference in its entirety. The XML file was created on Aug. 18, 2025, is named Sequence_Listing-010523, and has 34,400 bytes.

### BACKGROUND OF THE INVENTION

### Field of Invention

The instant disclosure provides multiple precision edits of bovine integrin beta 2 (CD18) that confer reduced susceptibility to Manheimia haemolytica leukotoxin A (LktA). Further disclosed are methods of producing genome-edited bovine cells and animals comprising a modified CD18 incorporating the precision edits.

### Background

Mannheimia haemolytica is a commensal bacterial species found in tonsillar crypts and the upper respiratory tracts of healthy cattle, and a major species associated with bovine respiratory disease complex (BRDC). Costing more than a billion dollars per year, this disease is a leading cause of morbidity, mortality, and economic loss in the beef and dairy cattle industries. (Whiteley et al, J. Vet. Intern. Med., (1992), 6:11-22; Griffin, D., Vet. Clin. North Am. Food Anim. Pract., (2010), 26:57-71)). BRDC typically begins with exposure to environmental stresses or co-infection with other viral or bacterial pathogens that can impair host defenses allowing M. haemolytica to proliferate and colonize the lungs where infection causes acute fibrinonecrotic pleuropneumonia (Griffin et al., Vet. Clin. North Am. Food Anim. Pract., (2010), 26:381-94; Singh et al, Vet. Pathol., (2011), 48:338-8). This bacterium expresses a variety of virulence factors that contribute to disease pathogenesis in the lungs. However, leukotoxin A (LktA) is the primary virulence factor contributing to the clinical signs and severe lung damage observed following infection (Highlander et al, Infect. Immun., (2000), 68:3916-22; Tatum et al, Microbiol. Pathogen., (1998), 24:37-46). Within hours of bacterial colonization of the lung, large numbers of polymorphonuclear leukocytes (PMN) infiltrate the airways (Whiteley, et al, supra; Ackerman et al, Microbes Infect., (2000), 2:1079-88). LktA binds to the bovine integrin beta-2 (CD18) subunit of the heterodimeric integrins (e.g., LFA1, FIG. 1) on the surface of PMN causing cell lysis and the release of pro-inflammatory cytokines, proteolytic enzymes, and reactive oxygen intermediates that intensify local inflammation (Singh et al, supra; Jeyaseelan et al, Anim. Health Res. Rev., (2002), 3:69-82; Deshpande et al, Infect. Immun., (2002), 70:5058-64; Dassanayake et al, Infect. Immun., (2007), 75:5004-10; Shanthalingam & Srikumarin, Proc. Nat'l. Acad. Sci., USA, (2009), 106:15448-53). Experimental depletion of PMN prior to infection (Slocombe et al, Am. J. Vet. Res., (1985), 46:2253-8), or infection with a LktA-deletion mutant of M. haemolytica (Highlander et al, supra; Tatum et al, supra) results in decreased morbidity and reduced lung lesions in calves. Thus, the interaction between the LktA toxin and its CD18 receptor is critical to the pathogenesis of M. haemolytica infection and is a potential intervention point for the prevention of disease.

In 2009, researchers reported the 22-amino acid signal peptide sequence of the CD18 remains uncleaved, and is the key region that binds to bacterial LktA (Shanthalingam & Srikumarin, supra). Normally, signal peptide sequences of mature Type I membrane proteins like CD18 are enzymatically hydrolyzed before their expression on the outer membrane of the cell. In ruminants, a conserved glutamine (Q) residue at position 18 apparently inhibits cleavage and thus the signal peptide remains with the mature CD18 protein where it can bind LktA and destroy the lymphocyte. Non-ruminant species are naturally resistant to LktA because their CD18 signal peptide undergoes cleavage due to a glycine (G) residue at position 18. Previous research showed leukocytes from a gene-edited CD18 cloned bovine fetus, encoding a G18-signal peptide, were unaffected by LktA exposure since the CD18 signal peptide was cleaved and unavailable for LktA binding (Shanthanlingam et al, Proc. Nat'l. Acad. Sci, USA, (2016), 113:13186-90). Thus, retention of the ruminant CD18 signal peptide appears to be the cause of LktA sensitivity in leukocytes.

However, it is possible that the above glycine edit at position 18 would be deleterious to ruminants since Q18 is perfectly conserved among all ruminants. However, live gene-edited calves with cleaved CD18 signal peptides have not been reported. Thus, the approach provided herein details modifications to CD18 that leave the CD18 signal peptide uncleaved, while substituting a single amino acid to disrupt LktA binding, and while maintaining the natural function of CD18.

### SUMMARY OF THE INVENTION

Disclosed herein are multiple synthetic CD18 signal peptides, the peptide comprising the sequence of any of SEQ ID NO: 3-38. Further disclosed herein are multiple synthetic CD18 proteins, the protein comprising a synthetic mutation in a signal peptide sequence having the sequence of any of SEQ ID NO:3-38. Further disclosed herein are bovine cells expressing any of the synthetic CD18 signal peptides or the synthetic CD18 proteins described herein. Further disclosed herein are isolated bovine cells expressing any of the synthetic CD18 signal peptides or the synthetic CD18 proteins described herein. Further disclosed herein is a bovine grown from an embryo, wherein the embryo comprises bovine cells expressing one of the synthetic CD18 protein described herein.

### INCORPORATION BY REFERENCE

All publications, patents and patent applications mentioned in this specification are herein incorporated by reference to the same extent as if each individual publication, patent or patent application was specifically and individually indicated to be incorporated by reference.

### BRIEF DESCRIPTION OF THE DRAWINGS

The novel features of the invention are set forth with particularity in the claims. Features and advantages of the present invention are referred to in the following detailed description, and the accompanying drawings of which:

FIG. 1 provides a depiction of an AlphaFold2 heterodimer model of LFA-1.

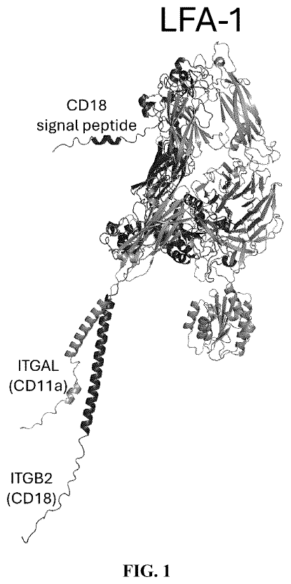

FIG. 2 provides a depiction of an in vitro LktA blocking assay. Prior to the introduction of the BL3 cells, synthetic CD18 signal peptides are mixed with the LktA preparation to block the toxin binding site.

FIG. 3 provides a depiction of the effect of serine substitution in synthetic CD18 signal peptides on LktA binding to CD18 in BL3 cells.

FIG. 4 provides a depiction of the reduction of M. haemolytica leukotoxin activity with synthetic peptides homologous to the bovine CD18. Synthetic 20-mer peptides were briefly preincubated with M. haemolytica LktA preparations as described in the Materials and Methods for the [BL3 cytotoxicity assay]. Each synthetic peptide differed from the common wild-type sequence (residues 2 to 21) by exactly one amino acid. The fold-change in toxin activity compared with the equivalent 20-mer wild-type sequence (LRQRPQLLLLAGLLALQSVL—(SEQ ID NO:1)). For these 400 synthetic peptides, results greater than 1.0 represent an increase in toxin binding (high susceptibility), while those less than 1.0 represent a decrease in toxin binding (lower susceptibility). The shaded cells denote the substitutions resulting in in 5- to 10-fold toxin resistance, “ti” denotes synthetic peptides that displayed toxin-independent cytotoxicity, and “sc” denotes peptides that consistently experienced synthesis failure.

FIG. 5 provides a representation of the characterization of CD18 surface expression and leukotoxin sensitivity in transduced P815 Cells. Surface expression levels of bovine CD18 in P815 cells, quantified by mean fluorescence intensity (MFI) from flow cytometry (left panel). In vitro sensitivity of transduced P815 cells to leukotoxin (Lkt) (right panel). For both panels, data are normalized to P815 cells expressing wild-type (WT) bovine CD18, which served as the positive control for leukotoxin susceptibility. As a negative control, P815 cells expressing bovine CD46 were also included.

FIG. 6 provides a representation of the relative inhibition of M. haemolytica LktA toxin activity by synthetic CD18 signal peptides with single residue substitutions. Based on results obtained by scanning 400 crude 20-mer synthetic CD18 signal peptides, a select group of highly-purified 22-mer CD18 signal peptides was synthesized. Serial dilutions of toxin preparations were preincubated with newly made signal peptides and added to bovine lymphocyte cells (BL3). The effect of toxin inhibition was normalized to that of the most common (wild type, WT) bovine CD18 signal peptide sequence. The mock sample had no toxin added. The error bars are the result of three experimental replicates on three different days.

### DETAILED DESCRIPTION OF THE INVENTION

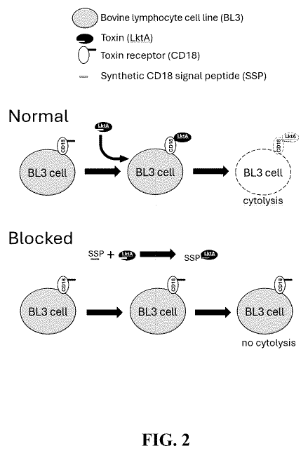

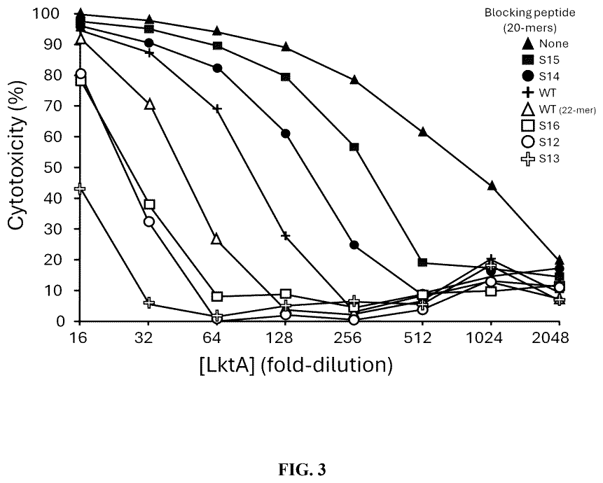

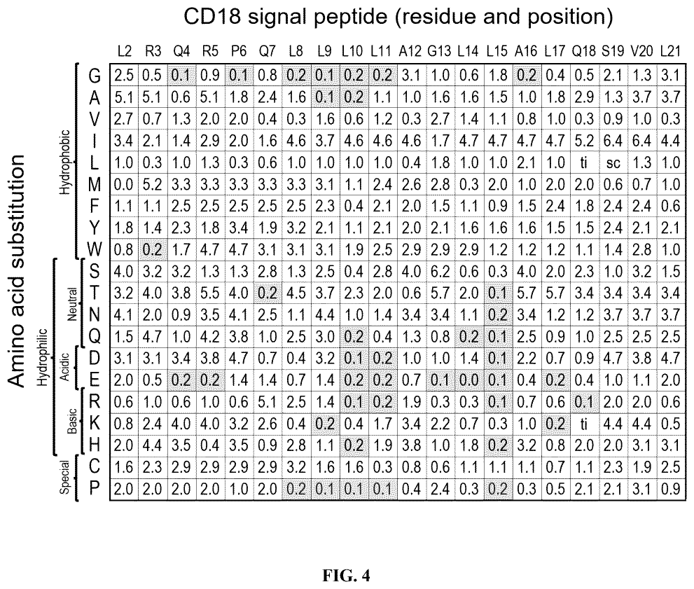

We have previously identified naturally occurring CD18 polypeptide sequence variation in cattle that alters the LktA-CD18 binding interaction and results in differences in lymphocyte sensitivity to M. haemolytica Lkt (Workman et al, F1000Research, (2018), 7:1985). We used an in vitro toxin blocking assay (FIG. 2) to discover these differences. The substitution of specific amino acids in synthetic CD18 signal peptides results in much tighter binding to LktA, when used in a LktS blocking assay with BL3 cells. The binding is specific and saturable. Thus, one way to significantly affect LktA binding is by altering the sequence of amino acids in the CD18 signal peptide. For example, systematically replacing each of the wildtype CD18 signal peptide residues at positions 12 through 16 with a serine, results in different levels of LktA blocking in the blocking assay depending on position (FIG. 3). We have used multiple distinct synthetic CD18 signal peptide sequences in in vitro LktA assays to identify CD18 sequences with significantly reduced LktA binding (FIG. 4). The variation was measured as the difference at 50% toxicity compared to no added blocking peptide and normalized to the wild type CD18 signal peptide sequence. This approach could offer a novel alternative to vaccination, which has not been broadly protective against LktA and BRDC. Moreover, even a partial reduction in sensitivity LktA could significantly reduce the need for antibiotic use in beef feedlots.

Vaccination can reduce the incidence and severity of BRDC in cattle, however, it provides incomplete protection and breakthrough infections still occur. Multiple meta-analysis reviews have found little compelling evidence that vaccines used near or at arrival at the feedlot reduce the incidence of BRD diagnosis. Conversely, antibiotics against the toxin-producing Pasturellacea bacteria can effectively prevent or reduce the impact of BRDC. However, the rise in antibiotic-resistance bacteria in cattle has, in some cases, rendered these chemotherapeutics useless. Together with the need to reduce the overall use of antimicrobials in livestock, novel prevention and control strategies would be desirable if available. One strategy to block toxin effects would be to disrupt its binding to the cellular receptor. Since bovine CD18 is the main cellular receptor for LktA in cattle, modifying the CD18 signal peptide sequence to reduce toxin binding offers a novel way to potentially reduce the burden of BRDC in cattle. Since BRDC is the most economically important infectious diseases affecting the cattle industry, if all cattle were unaffected by bacterial leukotoxin it would save the beef industry several billion dollars annually and drastically reduce the need for antibiotic use.

Preferred embodiments of the present invention are shown and described herein. It will be obvious to those skilled in the art that such embodiments are provided by way of example only. Numerous variations, changes, and substitutions will occur to those skilled in the art without departing from the invention. Various alternatives to the embodiments of the invention described herein may be employed in practicing the invention. It is intended that the included claims define the scope of the invention and that methods and structures within the scope of these claims and their equivalents are covered thereby.

Technical and scientific terms used herein have the meanings commonly understood by one of ordinary skill in the art to which the instant invention pertains, unless otherwise defined. Reference is made herein to various materials and methodologies known to those of skill in the art. Standard reference works setting forth the general principles of recombinant DNA technology include Sambrook et al., “Molecular Cloning: A Laboratory Manual”, 2d ed., Cold Spring Harbor Laboratory Press, Plainview, N.Y., 1989; Kaufman et al., eds., “Handbook of Molecular and Cellular Methods in Biology and Medicine”, CRC Press, Boca Raton, 1995; and McPherson, ed., “Directed Mutagenesis: A Practical Approach”, IRL Press, Oxford, 1991.

Any suitable materials and/or methods known to those of skill can be utilized in carrying out the instant invention. Materials and/or methods for practicing the instant invention are described. Materials, reagents and the like to which reference is made in the following description and examples are obtainable from commercial sources, unless otherwise noted. This invention teaches methods and describes tools for creating bovine cells and animals comprising precision edits to bovine CD18.

As used in the specification and claims, use of the singular “a”, “an”, and “the” include plural references unless the context clearly dictates otherwise.

The terms “isolated”, “purified”, or “biologically pure” as used herein, refer to material that is substantially or essentially free from components that normally accompany the referenced material in its native state.

The term “about” is defined as plus or minus ten percent of a recited value. For example, about 1.0 g means 0.9 g to 1.1 g and all values within that range, whether specifically stated or not.

The term “a nucleic acid consisting essentially of”, and grammatical variations thereof, means nucleic acids that differ from a reference nucleic acid sequence by 20 or fewer nucleic acid residues and also perform the function of the reference nucleic acid sequence. Such variants include sequences which are shorter or longer than the reference nucleic acid sequence, have different residues at particular positions, or a combination thereof.

The term “cattle” can generally refer to any member of the Bovinae subfamily and includes domesticated and wild cows, bulls, yak, bison, and buffalo.

### CD18 Precision Edits

The present disclosure provides methods of generating genetically modified bovine cells comprising precision edits to bovine CDIS. In some embodiments, endogenous nucleic acid sequences encoding CD18 protein are modified sch that at least one amino acid residue in the signal peptide of CD18 is substituted. Suchi substitutions result in both no cleavage of the CD18 signal peptide and a viable cell or animal and are used to generate genetically modified cells and animals derived therefrom. In some embodiments, the cells, tissues, organs and animals (e.g., bovids, cows) described herein comprise a modified CD18 coding sequence comprising an edit to the signal peptide sequence as listed in Table 1. The skilled artisan will recognize that where an individual peptidic is indicated in a particular sequence, the encoding nucleic acid can utilize any appropriate codon encoding the various amino acids.

TABLE 1
Exemplary CD18 Signal Peptide Sequences

| | | Residue |
|---|---|---|
| Sequence | SEQ ID NO: | change |
| MLRGRPQLLLLAGLLALQSVLS | SEQ ID NO: 3 | [G4] |
| MLRQRPQGLLLAGLLALQSVLS | SEQ ID NO: 4 | [G8] |
| MLRQRPQLGLLAGLLALQSVLS | SEQ ID NO: 5 | [G9] |
| MLRQRPQLLGLAGLLALQSVLS | SEQ ID NO: 6 | [G10] |
| MLRQRPQLLLGAGLLALQSVLS | SEQ ID NO: 7 | [G11] |
| MLRQRPQLLLLAGLLGLQSVLS | SEQ ID NO: 8 | [G16] |
| MLRQRPQLALLAGLLALQSVLS | SEQ ID NO: 9 | [A9] |
| MLRQRPQLLALAGLLALQSVLS | SEQ ID NO: 10 | [A10] |
| MLRQRPQLLLLAGLNALQSVLS | SEQ ID NO: 11 | [N15] |
| MLRQRPQLLQLAGLLALQSVLS | SEQ ID NO: 12 | [Q10] |
| MLRQRPQLLLLAGQLALQSVLS | SEQ ID NO: 13 | [Q14] |
| MLRQRPQLLLLAGLQALQSVLS | SEQ ID NO: 14 | [Q15] |
| MLRQRPQLLDLAGLLALQSVLS | SEQ ID NO: 15 | [D10] |
| MLRQRPQLLLDAGLLALQSVLS | SEQ ID NO: 16 | [D11] |
| MLRQRPQLLLLAGLDALQSVLS | SEQ ID NO: 17 | [D15] |
| MLRERPQLLLLAGLLALQSVLS | SEQ ID NO: 18 | [E4] |
| MLRQRPQLLELAGLLALQSVLS | SEQ ID NO: 19 | [E10] |
| MLRQRPQLLLEAGLLALQSVLS | SEQ ID NO: 20 | [E11] |
| MLRQRPQLLLLAELLALQSVLS | SEQ ID NO: 21 | [E13] |
| MLRQRPQLLLLAGELALQSVLS | SEQ ID NO: 22 | [E14] |
| MLRQRPQLLLLAGLEALQSVLS | SEQ ID NO: 23 | [E15] |
| MLRQRPQLLLLAGLLAEQSVLS | SEQ ID NO: 24 | [E17] |
| MLRQRPQLLRLAGLLALQSVLS | SEQ ID NO: 25 | [R10] |
| MLRQRPQLLLRAGLLALQSVLS | SEQ ID NO: 26 | [R11] |
| MLRQRPQLLLLAGRLALQSVLS | SEQ ID NO: 27 | [R14] |
| MLRQRPQLLLLAGLRALQSVLS | SEQ ID NO: 28 | [R15] |
| MLRQRPQLLLLAGLLAKQSVLS | SEQ ID NO: 29 | [K17] |
| MLRQRPQLLLLAGLLALKSVLS | SEQ ID NO: 30 | [K18] |
| MLRQRPQLLHLAGLLALQSVLS | SEQ ID NO: 31 | [H10] |
| MLRQRPQLLLLAGLHALQSVLS | SEQ ID NO: 32 | [H15] |
| MLRQRPQPLLLAGLLALQSVLS | SEQ ID NO: 33 | [P8] |
| MLRQRPQLPLLAGLLALQSVLS | SEQ ID NO: 34 | [P9] |
| MLRQRPQLLPLAGLLALQSVLS | SEQ ID NO: 35 | [P10] |
| MLRQRPQLLLPAGLLALQSVLS | SEQ ID NO: 36 | [P11] |
| MLRQRPQLLLLAGLPALQSVLS | SEQ ID NO: 37 | [P15] |
| MLRQRPQLLLLAGLTALQSVLS | SEQ ID NO: 38 | [T15] |
| MLRQRPQLLLLAGLLALGSVLS | SEQ ID NO: 39 | [G18] |

Cells, tissues, organs or animals can be genetically modified according to any method known by one of skill in the art. In some embodiments, the nucleic acid(s) in the cell are genetically modified such that one or more nucleic acid sequences (e.g., variant CD18 signal peptide sequences) in the cell are incorporated into the wild type CD18 gene. Nucleic acid sequences of the present disclosure can be genetically modified using any of the genetic modifications systems known in the art and/or disclosed herein. The genetic modification system utilized for practicing the instant disclosure can be a TALEN, a zinc finger nuclease, and/or a CRISPR-based system (such as CRISPR-Cas9).

Further disclosed herein is a blastocyst or an embryo cloned from a genetically modified cell comprising a precision edited version of the CD18 signal peptide sequence (e.g., Table 1). In such embodiments, the genetically modified nucleic acid(s) can be extracted from one genetically modified cell and cloned into a different cell. For example, in somatic cell nuclear transfer, the genetically modified nucleic acid from the genetically modified cell is introduced into an enucleated oocyte, Δn embryo of the present disclosure can be generated by fusing and activating the oocyte Such an embryo may be referred to herein as a “genetically modified embryo.” In some embodiments, the genetically modified embryo is transferred to the oviducts of a recipient female cow that has been made ready for pregnancy by synchronizing her estrus with that of the donor female.

A genetically modified blastocyst or embryo of the instant disclosure can be generated according to any technique known in the art. In some embodiments, the cells, blastocysts, embryos and the like that are generated according to any embodiment disclosed herein are tested for the presence of the precision edited CD18 signal peptide sequence, Any technique used in the art can be applied in the context of the instant disclosure. Non-limiting examples of such techniques include: Miseq and Sanger sequencing to detect gene targeting efficiency; exome sequencing and/or whole genome sequencing to detect off-targeting; karyotyping to detect chromosome abnormalities; RT-qPCR to detect target gene expression; RNAseq to detect the normality of whole gene expression pattern; specific gene antibody binding: blastocyst development ratio to check whether gene editing influences embryo development, and any combination thereof.

A genetically modified blastocyst or embryo of the instant disclosure can be transferred to a surrogate, for example, into the oviduct of the surrogate. If desired, more than one blastocyst or embryo can be transferred to a surrogate. Embryos or blastocysts derived from a sample cell can be transferred into multiple surrogates. In some embodiments, at least one wild-type blastocyst or embryo is transferred to the surrogate at the same time or substantially the sane time as the genetically modified blastocyst or embryo. The surrogate can be checked for pregnancy an appropriate number of days after transfer of the genetically modified blastocyst or embryo. Genetic testing can be utilized to confirm the presence of the desired CDIS variant in any blastocyst or embryo. Genetically modified blastocysts or embryos can be grown into a post-natal genetically modified animal (e.g., bovid, cow), including neonatal, juvenile, adult, female, and male post-natal animals. A genetically modified animal of the instant disclosure can be bred With a Non-Genetically Modified Animal or Another Genetically Modified Animal.

### CRISPR Technology

For some embodiments CRISPR-based technology is utilized to construct the genetically modified cells and animals disclosed herein. In some embodiments, a CRISPR-based agent is one or more polynucleotides involved in the expression of or directing the activity of CRISPR-associated (“Cas”) genes, including, but not limited to, sequences encoding a Cas gene, a “tracr” (trans-activating CRISPR) sequence (e.g., tracrRNA or an active partial tracrRNA), a tracr-mate sequence, a guide (“spacer”) sequence, and/or other sequences and transcripts from a CRISPR locus. In some embodiments, the CRISPR-based agent is a polynucleotide that encodes at least one CRISPR protein and one or more guide RNAs (gRNAs). The one or more gRNAs can comprise a sequence cognate to a modified CD18 polynucleotide sequence and capable of binding to a protospacer adjacent motif (“PAM”).

Some embodiments described herein can utilize a CRISPR-based polypeptide or fragment or derivative thereof that targets one or more CD18 DNA sequences in a cell. In some embodiments, the CRISPR-based agent is one or more CRISPR/Cas endonuclease or biologically active fragments or derivatives thereof, or one more nucleic acids encoding one or more CRISPR/Cas polypeptides or fragments or derivatives thereof. In some embodiments, the CRISPR/Cas endonuclease or a derivative thereof is from the CRISPR Type I, Type II, Type IIA, Type IIC, Type III, Type IV, Type V, Type VI, Type IIA, Type JIB, or Type HC system. The skilled artisan can select an appropriate CRISPR system to practice the methodologies described herein.

CRISPR-based agent typically comprises a gRNA. In some embodiments, the gRNA targets a nucleotide sequence encoding a bovine CD18 signal peptide. In some embodiments of the present invention, the gRNA comprises any nucleotide sequence corresponding to the modified CD18 precision edit (see, e.g., Table 1).

The process of converting a functional discovery in a cell line into a healthy gene-edited calf expressing a desirable trait is a multistep process that starts with knowledge of the molecular/gene/protein functions. Once the target and specific variant are identified, the genomic DNA sequence of the immortalized cell line can be used to design a guide RNA that, with CRISPR tools and technology, cuts the genome uniquely at that site in the parent cell line. Simultaneously, a DNA repair template is provided that encodes the desired nucleotide alteration, usually encoding a new amino acid. After isolation and verification of the correct daughter cell line edits, they are tested for correct expression of the desirable trait. In our specific case, the level of LktA resistance expressed by the edited daughter cell line is expected to correspond to that of the original blocking peptide used with the parent cell line. It is expected that cells in an edited calf with the same edit will also show the same trait. In some cases, the trait was more clearly pronounced in the fetus or live calves, that it was in the cell line (Workman et al, (2023), supra).

The next milestone includes, editing a cell line that can be used to make a viable embryo. Historically adult bovine fibroblast primary cell lines have been used. These can be collected from a simple ear biospy, amplified in culture, and frozen. They have the advantage of being easy to collect and wide available. However, they have limited division capabilities (about 30 doublings total), and low success rate for producing calves (10% or worse). More recently, ESCs made from blastocysts have been developed that are not limited in their capacity to divide and more pluripotent. Regardless of whether adult fibroblasts or ESCs are used, they are edited like the original immortalized cell lines, screened for the correct edit, and expanded in culture.

The nuclei of these cells are then transferred to a fresh oocyte that has been enucleated and proprietary electrical and chemical treatments are used to stimulated embryonic development. The 7-day edited blastocysts are then transferred individually to estrus-synchronize recipient cows. At approximately 30 days post embryo transplantation, ultrasound is used to confirm pregnancy, and at 60 days ultrasound may be used to confirm the fetal sex. At approximately 282 days of embryo development the calves may be born naturally or be cesarian section.

The embodiments illustratively disclosed herein suitably may be practiced in the absence of any element [e.g., method (or process) steps or composition components)] which is not specifically disclosed herein. Thus, the specification includes disclosure by silence. Written support for a negative limitation may also be found through the absence of the excluded element in the specification, known as disclosure by silence.

Having generally described this invention, the same will be better understood by reference to certain specific examples, which are included herein to further illustrate the invention and are not intended to limit the scope of the invention as defined by the claims.

### EXAMPLES

### Example 1

Preparation of M. haemolytica Leukotoxin

LktA was produced from a field strain of M. haemolytica (USDA-ARS-USMARC-183 serotype A1) isolated from an animal that was part of a high-mortality respiratory disease outbreak in a Kansas feedlot in 1991 (Workman et al., (2018), supra). The isolate was maintained on Brain Heart Infusion (BHI) agar (Sigma-Aldrich) and frozen stocks were stored in BHI broth with 20% glycerol at −80° C. LktA was partially purified from batch liquid cultures as previously described (Workman et al., (2018), supra). Briefly, a single, 24-hour colony isolate from BHI agar was inoculated into 5 ml BHI broth in a 10 ml culture tube and incubated overnight at 37° C. in 5% CO₂ without shaking. The following morning, 1 ml of BHI liquid culture was inoculated into 100 ml of a fresh semi-defined medium 2 (SDM2, (van Rensburg & du Preez, J. App. Microbiol., (2007), 102:1273-82)) fresh culture medium in a 300 ml Delong-style Erlenmeyer flask with baffles (Corning, Inc., Corning, NY, USA) and incubated at 37° C., 250 rpm, in 5% CO₂. The SDM2 is an amino acid-limited culture medium supplemented with cysteine, glutamine, ferric iron, and manganese and greatly improves Lkt production in aerobic batch culture (van Rensburg & du Preez, supra). Bacterial cell growth was monitored optically by turbidity at 600 nm until the transition between log and stationary phase growth was evident. At that point, the flask was rapidly chilled on ice with shaking and the culture was centrifuged at 39,000×g for 8 min at 4° C. in angle bucket rotor with 35 ml round bottom, high-speed, clear, polycarbonate centrifuge tubes. The clarified supernatant was immediately decanted, flash frozen in liquid nitrogen, and stored at −80° C. until use. The LktA of clarified SDM2 culture supernatants was estimated to be 90% pure based on gel densitometry imaging of coomassie-stained SDS-PAGE gels.

For long term storage, glycerol and HEPES buffer (2-[4-(2-hydroxyethyl)piperazin-1-yl]ethanesulfonic acid) were added to final concentrations of 40% and 100 mM (pH 7.55), respectively. The biological activity of LktA in this preparation was stable at −80° C. for more than ten years without noticeable loss, when measured by cytolytic and cytotoxic assays. The frozen, buffered LktA was diluted approximately 512-fold to achieve 50% killing of BL3 cells grown in RPMI for 48 hours and suspended at a density of 6×10⁵ cells/ml.

### Synthesis and Use of CD18 Synthetic Signal Peptides

Custom bovine CD18 signal peptides were commercially synthesized as 22-mers (GenScript Biotech Corporation, Piscataway, NJ), purified to greater than 95% purity by preparative high-performance liquid chromatography, and lyophilized. The wild-type synthetic signal peptide (propeptide residues 1-22: MLRQRPQLLLLAGLLALQSVLS (SEQ ID NO:2)) was dissolved in dimethysulfoxide (DMSO) at a concentration of 10 mg/ml (4 mM), aliquoted, and stored at −20° C. until use. For use in LktA toxicity assays, aliquots of the 4 mM peptides solutions in DMSO were diluted in RPMI to a 64 μM working solution and preincubated with LktA preparations as described below. For high throughput screening of synthetic CD18 signal peptides, the peptides were commercially synthesized as 20-mers (GenScript Biotech Corporation, Piscataway, NJ), purified to greater than 70% purity by preparative high-performance liquid chromatography, and lyophilized. The wild-type synthetic signal peptide (propeptide residues 2-21: LRQRPQLLLLAGLLALQSVL (SEQ ID NO:1). Thus, these peptides were 1 residue shorter at either end of the signal peptide. Previous studies had shown these two amino acids are not critical for LktA binding (Workman et al., (2018), supra). After all 400 of the 20-mer peptides with reduced LktA binding properties were identified, the corresponding 22-mer peptides were made at 95% purity and retested to confirm their properties.

### Cytolytic, Cytotoxic, and Peptide Blocking Assays

Cell viability after LktA exposure was measured by methods similar to those previously described (Shanthalingam & Srikumaran, supra; Workman et al, (2018), supra). The primary difference was replacing the NADH/NADPH-dependent reduction of tetrazolium (MTT, (3-(4,5-dimethylthiazol-2-yl)-2,5-diphenyl-2H-tetrazolium bromide) into formazan crystals, with the ATP-dependent oxidation of luciferin with luciferase (CellTiter-Glo® 2.0 Assay, Promega Corporation). This chemiluminescent cell viability assay detects the amount of APT present thus indicating metabolically active cells. We also measured the lack of membrane integrity (cytolysis) with a non-toxic asymmetric cyanine dye that is excluded from viable cells, but binds DNA released from lysed cells and fluoresces proportionally (CellTox™ Green Cytotoxicity Assay, Promega Corporation Madison, Wisconsin). The fluorescent dye of the cytolysis assay could also be multiplexed with the chemiluminescent cell viability assay as needed. Cultures of actively growning BL3 cells grown in RPMI were washed in PBS and seeded to a density of 5×10⁵ cells/ml. The cells were incubated for 48 hours, washed once in PBS, and suspended in colorless RPMI without serum at a density of 6×10⁶ cells/ml.

LktA activity was titrated with BL3 cells in a 96-well microplate for fluorescence and chemiluminescence detection (HARD-SHELL, Bio-Rad, Hercules, California). The first set of wells were loaded with 100 μl of a 1:8 LktA preparation diluted in colorless RPMI (without serum). Two-fold serial dilutions were made by removing 50 μl of the 1:8 LktA preparation and diluting is subsequent wells with 50 μl of the same media. Finally, a 50 μl aliquot of the BL3 cells was added to each well so that the most concentrated well with LktA had a final dilution of 1:16 from the original toxin preparation. The 100 μl assay mixtures in 96-well plates were incubated for 1 hr at 37° C. in 5% CO₂.

When synthetic CD18 signal peptides were used to block LktA activity, 32 μM the peptide solutions (amino acids 1-22) were pre-incubated with toxin preparations for 30 minutes at room temperature prior was preloaded with 50 μl of the same medium as the cells and two-fold serial dilutions of an LktA preparation to the addition of BL3 cells and the 100 μl assay mixture was incubated for 1 hr at 37° C. in 5% CO₂. After the 1 hr incubation of cells and toxin, a 20 μl solution of a cyanine dye was added to each well and fluorescence intensity was measured at 485 nm excitation and 520 nm emission on a multiple mode microplate reader (SpectraMax M5, Molecular Devices, LLC, San Jose, California) and recorded as relative fluorescence units (RFU).

Background fluorescence was estimated from BL3 wells without LktA preparations added and defined as zero percent lysis. Maximum cytolysis was defined as 100 percent lysis and was estimated by measuring the fluorescence in control wells where 4 μl of a lysis buffer was added at the beginning of the 60-minute incubation. The percent cytolysis for a given LktA dilution was calculated as follows: (LktA dilution RFU−background RFU)/(lysis control RFU −background RFU).

Cytotoxicity could be measured in the same well with a chemiluminescent cell viability assay that detects the amount of APT present thus indicating metabolically active cells per the manufacturer's instruction (The CellTiter-Glo® 2.0 Assay, Promega Corporation Madison, Wisconsin). After collecting fluorescence measurements, 40 μl of the chemiluminescent reagent was added to each well and mixed by pipette to facilitate ATP extraction from the cells. Chemiluminescence was measured for 5 minutes using a multiple mode microplate reader (SpectraMax M5, Molecular Devices, LLC) and recorded as relative chemiluminescence units (RCU).

Background chemiluminescence was measured from the chemiluminescence of the BL3 lysis control wells where 4 μl of a lysis buffer was added at the beginning of the 60-minute incubation. Minimum cytotoxicity was defined as zero percent and estimated from the chemiluminescence of BL3 wells without LktA preparations added. The percent cytotoxicity for a given LktA dilution was calculated as follows: 1−[(LktA dilution RLU−background RLU)/(BL3 no LktA RLU−background RLU)].

Summary of results: Specific, single amino acid substitutions were made in synthetic wild-type CD18 signal peptides and demonstrated significant reductions in their binding to M. haemolytica LktA toxin (FIG. 3). First, we did this with approximately 12 highly purified (>95% purity) 22-mers each having a different single amino acids substitution. Next, we used high-throughput commercial 20-mer peptide synthesis to screen every possible single amino acid substitution (20) against every possible CD18 signal peptide position, from 2 to 21. Of these 400×20-mer synthetic CD18 peptides screened, we found that key positions and key residues that interfere with the normal CD18 binding to the toxin (FIG. 4). Compared to the wild-type unsubstituted synthetic CD18 signal peptide, some single amino acid substitutions reduce the synthetic peptide binding by as much as 10-fold, while others increase the peptide binding by as much as 6-fold. The dynamic range in these assays spans six doublings. There are multiple single amino acid substitution 400 peptides that caused at least a two-fold reduction in toxin sensitivity, including several peptides that caused a 10-fold reduction (FIG. 4, values <0.5 and <0.1, respectively). In addition, CRISPR-edited CD18 knockout mutants of the BL3 cell line have been created and are being grown in culture for testing for LktA resistance. These will be used for negative controls to compare with CRISPR-edited BL3 cell lines halving single amino acid CD18 signal peptide variants (like those in Table 1). The LktA resistance measured in CD18-edited BL3 cell lines will determine which peptide variants are targeted for production of live calves with reduced susceptibility to M. haemolytica LktA toxin.

### Example 2

### Mouse Model of Bovine CD18

Cells: The cell lines 293T (ATCC CRL-3216, human kidney) and P815 (ATCC TIB-64, murine mastocytoma) were propagated in DMEM (Corning), while BL3 (bovine lymphoma, kindly provided by Dr. Subramaniam Srikumaran) were propagated in RPMI 1640 medium (Cytiva HyClone). All media were supplemented with 10% serum and 1× antibiotic-antimycotic (Gibco). For specific serum details, 293T cells utilized U.S. origin FBS (Peak Serum, Catalog no. PS-FB1). P815 cells were grown with gamma-irradiated EquaFetal serum (Atlas Biologicals, Catalog no. EF-0500-A). BL3 cells were grown with gamma-irradiated U.S. origin FBS (Cytiva Hyclone, Catalog no. SH30541.03).

Lentivirus Production: 293T cells were seeded in 10 cm plates at a density calculated to achieve 70-80% confluence the following day. The following day, 293T cells were transfected with lentivirus plasmids using Lipofectamine 3000 Reagent (Thermo Fisher Scientific). For each 10 cm plate, a total of 20 μg of plasmid DNA was used, consisting of 4 μg pMD2.G, 3 μg pRSV-REV, 3 μg pMDLg/pRRE, and 10 μg of a transfer vector containing our gene of interest.

Prior to transfection, the cell culture medium was replaced with 9 mL of fresh DMEM supplemented with 10% FBS and 1× GlutaMAX (no antibiotic-antomycotic). The DNA-lipid complex (1 mL) was added dropwise to the cells, and plates were incubated overnight at 37° C. with 5% CO₂.

Approximately 16 hours post-transfection, the media was removed and gently replaced with 10 mL DMEM supplemented with 10% FBS, 1× GlutaMAX and 5 mM sodium butyrate. Cells were incubated for 8 hours at 37° C. with 5% CO₂. Following this, the media was changed to 5.5 mL of DMEM supplemented with 10% FBS, 1× GlutaMAX, and 10 mM HEPES, and cells were incubated for an additional 24 hours at 37° C. with 5% CO₂.

Lentiviral supernatant was collected approximately 24 hours after the HEPES media change. Supernatant (approximately 5 mL per 10 cm plate) was transferred to 15 mL conical tubes on ice and filtered through a 0.45 μm syringe filter. The filtered lentivirus (approximately 4.5 mL) was then added to 100 kDa 15 mL Amicon Ultra centrifugal filter units and concentrated by centrifugation at 3,200×g for 10 minutes at room temperature. The concentrated lentivirus was carefully collected and 1 μL of polybrene was added per 1 mL of concentrated lentivirus.

Spinfection of P815 Cells: P815 cells were spinfected with the concentrated lentivirus. Approximately 4×10⁵ P815 cells in complete growth medium were combined with 1 mL of the appropriate lentivirus prep in 15 mL conical tubes, resulting in a 2 mL final volume. Cells were spinfected at 800× g for 30 minutes at room temperature. Following spinfection, cells were resuspended, transferred to 6-well plates, and incubated overnight at 37° C. with 5% CO₂.

Puromycin Selection of Lentivirus Transduced Cells: Approximately two days post-spinfection, puromycin selection was initiated. Cell media (approximately 2 mL per well) was collected in 50 mL conical tubes. Cells were washed once with 1 mL Phosphate-Buffered Saline (PBS). TrypLE Express was added to each well, and cells were incubated at 37° C. for approximately 2 minutes to detach. TrypLE Express was neutralized with complete media, and cells were collected. Cell pellets were spun down at 300× g for 5 minutes at 4° C. Supernatant was removed, and cell pellets were resuspended in 2 mL of DMEM supplemented with 10% FBS, lx antibiotic-antimycotic, and 2 μg/mL puromycin. Cell counts were performed using a Countess II automated cell counter with Trypan Blue. Cell volumes were adjusted to 2×10⁵ cells/mL in complete growth medium containing 2 μg/mL puromycin and transferred to T-25 flasks. Flasks were rocked gently and incubated overnight at 37° C. with 5% CO₂. Transduced P815 cells were subcultured every 2-3 days in DMEM supplemented with 10% FBS, lx antibiotic-antimycotic, and 2 μg/mL puromycin.

Flow Cytometric Analysis of CD18 Surface Expression: To measure surface expression of CD18, 2×10⁵ BL3 or P815 cells were fixed with 4% paraformaldehyde (PFA) in PBS for 12 minutes at room temperature. Cells were pelleted at 400× g for 2 minutes and washed three times with PBS. Cells were blocked using 2% bovine serum albumin (BSA) diluted in PBS (BSA-PBS) for 30 minutes at room temperature. Cells were pelleted at 400× g for 2 minutes and resuspended in 1% BSA-PBS containing 10 μg/mL of a mouse anti-bovine CD18 antibody (clone BAQ30A; Invitrogen, Waltham, MA) and incubated 30 minutes at room temperature. The cells were pelleted at 400× g for 2 minutes, washed three times with PBS, and resuspended with 1% BSA-PBS containing 0.5 μg/mL of a mouse IgG Fc binding protein conjugated to CruzFluor 488 (Santa Cruz Biotechnology; Dallas, TX) and incubated for 30 minutes on ice in the dark. Cells were pelleted at 400× g for 2 minutes, washed twice with PBS, and resuspended in PBS for analysis. Cells were analyzed using an Attune NxT Flow Cytometer (ThermoFisher Scientific) using the Attune cytometric software version 5.1.1.

P815 Cell Model and Experimental Design: Our experimental model used P815 cells, a murine (mouse) mastocytoma cell line. We chose P815 cells because, although they are a murine immune cell type (analogous to the bovine immune cells that are a natural target of leukotoxin [Lkt]), they are intrinsically resistant to Lkt. This resistance stems from the natural cleavage of their endogenous CD18 signal peptide, which prevents Lkt binding (Shanthalingam & Srikumaran, Proc. Nat'l. Acad. Sci., (2009), 106:15093-98). Furthermore, previous studies showed that expressing bovine CD18 in otherwise Lkt-resistant murine cell lines, including P815, is enough to make them Lkt sensitive (Deshpande et al., Infect. Immun., (2002), 70:5058-64; Shanthalingam & Srikumaran, supra). It has been shown that bovine CD18 can functionally heterodimerize with murine CD11a on the cell surface, forming a complete beta-2 integrin receptor complex (Deshpande et al., supra). This established system provides an in vitro platform to investigate how specific amino acid changes within the bovine CD18 signal peptide impact its expression and functional interaction with leukotoxin.

To investigate leukotoxin susceptibility, we transduced P815 cells with lentivirus to express either wild-type (WT) bovine CD18 or CD18 variants containing single amino acid substitutions. As a negative control, we also transduced P815 cells with lentivirus expressing bovine CD46, a protein not expected to confer leukotoxin susceptibility. We then measured bovine CD18 surface expression using flow cytometry and assessed Lkt sensitivity through in vitro toxicity assays. All reported values are normalized to P815 cells expressing WT bovine CD18.

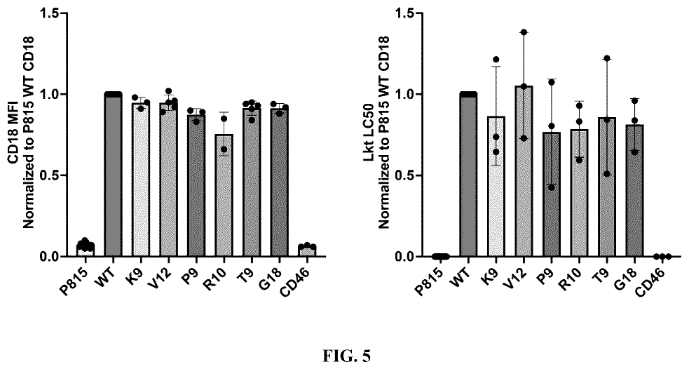

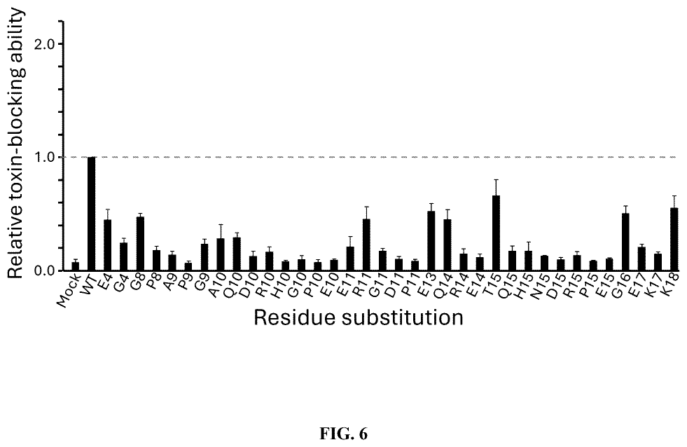

Functional Characterization of CD18 Variants: Based on the prior 20-mer signal peptide blocking studies (FIG. 4), and the replicated studies with highly-purified 22-mer (full length) signal peptide blocking studies (FIG. 6), we anticipated CD18 P9 [Seq ID No: 34] and R10 [Seq ID No: 25] variants would exhibit low LktA binding and susceptibility. Conversely, we anticipated K9, V12, and T9 variants would have LktA binding and sensitivity similar to WT CD18 (FIG. 4). However, contrary to these predictions, comparative analysis revealed no significant differences in either CD18 surface expression or Lkt sensitivity among P815 cells transduced with WT CD18 versus those expressing any of the tested CD18 variants. As anticipated, untransduced P815 cells and those expressing bovine CD46, which served as negative controls, lacked bovine CD18 expression and were resistant to Lkt (FIG. 5). Note that this is confounded by the mouse system which expresses mouse CD18, mouse signal peptide cleavage enzymes, and mouse heterdimeric partners for bovine CD18.

The G18 (SEQ ID NO: 39) variant, previously shown by others to render bovine cells resistant to Lkt by making the CD18 signal peptide cleavable, was included as a control in our study (Shanthalingam & Srikumaran, supra; Shanthalingam et al., Scient. Rep., (2016), 6, 37533). Unexpectedly, this variant also exhibited no reduction in Lkt susceptibility in our P815 cell model (FIG. 5). It's currently unclear whether the G18 (SEQ ID NO:39) variant's signal peptide is cleaved as expected within this murine system, or if the primary Lkt binding site is outside the signal peptide domain in this experimental context. It's possible the mechanism mediating Lkt susceptibility in this mouse model differs from that observed in bovine cells.

### Example 3

Leukotoxin Inhibition Assay Replication with Synthetic Highly Purified 22-Mer CD18 Signal Peptides

To further test the reproducibility of the peptide-toxin binding, a selection of the most promising 20-mer synthetic peptides (70% pure) identified in the first round of toxin blocking assays (FIG. 4) were re-synthesized as corresponding, full-length 22-mers at 95% purity (FIG. 6). Other than a difference in peptide length and purity, the blocking experiments were performed as described above in Example 1. The peptide sequences are listed in Table 1, above.

Substituted 22-mers with very low toxin inhibition are hypothesized to have low toxin binding, and their substitute residues are candidates for gene editing of CD18 in cell lines or animals. Among the most dramatic and reproducible low-toxin-binding peptides were those in the leucine-rich alpha-helical portion of the signal peptide (positions 8-11, 14, 15, and 17). These included single amino acid substitutions with charged side chains (acidic and basic) and those affecting rotation about the alpha carbon in the peptide backbone (glycine and proline)

### Example 4

### Method of Producing CD18-Edited Calves

To generate live calves harboring a specific CD18 genetic modification, embryonic stem cell nuclear transfer will be employed. Donor cattle exhibiting superior genetic traits will be selected to provide gametes (sperm and eggs) for in vitro fertilization (IVF). Fertilized oocytes (eggs) will be developed into blastocysts (early-stage embryos) under controlled laboratory conditions. Embryonic stem cells (ESCs, undifferentiated cells capable of developing into any cell type) will be derived from the inner cell mass of these blastocysts. CRISPR/Cas9-mediated genome editing will be applied to introduce the desired CD18 modification into the ESCs. Comprehensive genomic analysis (whole genome sequencing) will be performed to verify the accuracy of the edit and to exclude unintended genetic alterations. Subsequently, ESC nuclear transfer will be conducted to produce cloned embryos carrying the modified CD18 gene. This process involves enucleation of oocytes (removal of the genetic material) followed by fusion with a CD18-edited ESC. The reconstructed oocyte will be artificially activated to initiate in vitro embryonic development. Embryos exhibiting optimal developmental characteristics will be transferred into surrogate females (recipient cows) to establish pregnancies.

Alternatively, one-cell stage embryos (zygotes) can be genetically modified to introduce a specific CD18 alteration, potentially resulting in live animals carrying the desired modification. Following in vitro fertilization (IVF), the CRISPR-Cas9 gene editing system and a corresponding DNA repair template can be introduced into the zygote via electroporation or microinjection. These embryos will be cultured in vitro to early developmental stages before transfer into surrogate recipient cows for gestation.

While the invention has been described with reference to details of the illustrated embodiments, these details are not intended to limit the scope of the invention as defined in the appended claims. The embodiment of the invention in which exclusive property or privilege is claimed is defined as follows:

What is claimed is:

1. A synthetic CD18 signal peptide, the peptide comprising the sequence of any of SEQ ID NO: 3-38.

2. A synthetic CD18 protein, the protein comprising a synthetic mutation in a signal peptide sequence, the signal peptide comprising the sequence of any of SEQ ID NO:3-38.

3. A bovine cell expressing the synthetic CD18 signal peptide of claim 1, or the synthetic CD18 protein of claim 2.

4. An isolated bovine cell expressing the synthetic the synthetic CD18 signal peptide of claim 1, or the synthetic CD18 protein of claim 2.

5. A bovine grown from an embryo, wherein the embryo comprises bovine cells expressing the synthetic CD18 protein of claim 2.


SEQUENCE LISTING (39 sequences)

SEQ ID NO: 1 - protein, 20 aa, Bos taurus
LRQRPQLLLLAGLLALQSVL

SEQ ID NO: 2 - protein, 22 aa, Bos taurus
MLRQRPQLLLLAGLLALQSVLS

SEQ ID NO: 3 - protein, 22 aa, synthetic construct
MLRGRPQLLLLAGLLALQSVLS

SEQ ID NO: 4 - protein, 22 aa, synthetic construct
MLRQRPQGLLLAGLLALQSVLS

SEQ ID NO: 5 - protein, 22 aa, synthetic construct
MLRQRPQLGLLAGLLALQSVLS

SEQ ID NO: 6 - protein, 22 aa, synthetic construct
MLRQRPQLLGLAGLLALQSVLS

SEQ ID NO: 7 - protein, 22 aa, synthetic construct
MLRQRPQLLLGAGLLALQSVLS

SEQ ID NO: 8 - protein, 22 aa, synthetic construct
MLRQRPQLLLLAGLLGLQSVLS

SEQ ID NO: 9 - protein, 22 aa, synthetic construct
MLRQRPQLALLAGLLALQSVLS

SEQ ID NO: 10 - protein, 22 aa, synthetic construct
MLRQRPQLLALAGLLALQSVLS

SEQ ID NO: 11 - protein, 22 aa, synthetic construct
MLRQRPQLLLLAGLNALQSVLS

SEQ ID NO: 12 - protein, 22 aa, synthetic construct
MLRQRPQLLQLAGLLALQSVLS

SEQ ID NO: 13 - protein, 22 aa, synthetic construct
MLRQRPQLLLLAGQLALQSVLS

SEQ ID NO: 14 - protein, 22 aa, synthetic construct
MLRQRPQLLLLAGLQALQSVLS

SEQ ID NO: 15 - protein, 22 aa, synthetic construct
MLRQRPQLLDLAGLLALQSVLS

SEQ ID NO: 16 - protein, 22 aa, synthetic construct
MLRQRPQLLLDAGLLALQSVLS

SEQ ID NO: 17 - protein, 22 aa, synthetic construct
MLRQRPQLLLLAGLDALQSVLS

SEQ ID NO: 18 - protein, 22 aa, synthetic construct
MLRERPQLLLLAGLLALQSVLS

SEQ ID NO: 19 - protein, 22 aa, synthetic construct
MLRQRPQLLELAGLLALQSVLS

SEQ ID NO: 20 - protein, 22 aa, synthetic construct
MLRQRPQLLLEAGLLALQSVLS

SEQ ID NO: 21 - protein, 22 aa, synthetic construct
MLRQRPQLLLLAELLALQSVLS

SEQ ID NO: 22 - protein, 22 aa, synthetic construct
MLRQRPQLLLLAGELALQSVLS

SEQ ID NO: 23 - protein, 22 aa, synthetic construct
MLRQRPQLLLLAGLEALQSVLS

SEQ ID NO: 24 - protein, 22 aa, synthetic construct
MLRQRPQLLLLAGLLAEQSVLS

SEQ ID NO: 25 - protein, 22 aa, synthetic construct
MLRQRPQLLRLAGLLALQSVLS

SEQ ID NO: 26 - protein, 22 aa, synthetic construct
MLRQRPQLLLRAGLLALQSVLS

SEQ ID NO: 27 - protein, 22 aa, synthetic construct
MLRQRPQLLLLAGRLALQSVLS

SEQ ID NO: 28 - protein, 22 aa, synthetic construct
MLRQRPQLLLLAGLRALQSVLS

SEQ ID NO: 29 - protein, 22 aa, synthetic construct
MLRQRPQLLLLAGLLAKQSVLS

SEQ ID NO: 30 - protein, 22 aa, synthetic construct
MLRQRPQLLLLAGLLALKSVLS

SEQ ID NO: 31 - protein, 22 aa, synthetic construct
MLRQRPQLLHLAGLLALQSVLS

SEQ ID NO: 32 - protein, 22 aa, synthetic construct
MLRQRPQLLLLAGLHALQSVLS

SEQ ID NO: 33 - protein, 22 aa, synthetic construct
MLRQRPQPLLLAGLLALQSVLS

SEQ ID NO: 34 - protein, 22 aa, synthetic construct
MLRQRPQLPLLAGLLALQSVLS

SEQ ID NO: 35 - protein, 22 aa, synthetic construct
MLRQRPQLLPLAGLLALQSVLS

SEQ ID NO: 36 - protein, 22 aa, synthetic construct
MLRQRPQLLLPAGLLALQSVLS

SEQ ID NO: 37 - protein, 22 aa, synthetic construct
MLRQRPQLLLLAGLPALQSVLS

SEQ ID NO: 38 - protein, 22 aa, synthetic construct
MLRQRPQLLLLAGLTALQSVLS

SEQ ID NO: 39 - protein, 22 aa, synthetic construct
MLRQRPQLLLLAGLLALGSVLS